(19) United States
(12) Patent Application Publication
(10) Pub. No.: US 2009/0240086 A1
(43) Pub. Date: Sep. 24, 2009

(54) Preparation of glycerol tert-butyl ethers

(76) Inventors: Edward A. Barsa, Glen Mills, PA (US); Beth M. Steinmetz, Bryn Mawr, PA (US)

Correspondence Address: LyondellBasell Industries, 3801 WEST CHESTER PIKE, NEWTOWN SQUARE, PA, 19073, US

(21) Appl. No.: 12/077,252

(22) Filed: Mar. 18, 2008

(51) Int. Cl. C07C 41/06

(52) U.S. Cl. 568/671

(57) ABSTRACT

A process for making glycerol di-tert-butyl ethers is disclosed. In one aspect of the invention, glycerol and isobutylene react in the presence of a β-zeolite having a silicon to aluminum ratio greater than 150. In another aspect, the etherification is performed in the presence of a β-zeolite and added tert-butyl alcohol. Each process selectively provides glycerol di-tert-butyl ethers while reducing the generation of isobutylene dimers and trimers. Utilizing both aspects of the inventive process simultaneously affords a diether product mixture containing less than 5 wt. % of isobutylene oligomers.

### FIELD OF THE INVENTION

The invention relates to an improved way to make tert-butyl ethers of glycerol, particularly glycerol di-tert-butyl ethers. The ethers are valuable fuel additives.

### BACKGROUND OF THE INVENTION

As crude oil prices soar, the world labors to identify practical fuels based on renewable resources. Biodiesel, produced by triglyceride methanolysis, is a popular alternative. Thus, vegetable or animal oils are transesterified with methanol using base catalysis to a mixture of fatty methyl esters, which can be used for fuel. For every 10 kilograms of biodiesel produced, 1 kilogram of glycerol is generated as a by-product. Because glycerol is not compatible with biodiesel or other fuels, it must be separated out and used elsewhere. Unfortunately, glycerol has limited utility. Moreover, expensive refining steps are needed to convert crude glycerol into commercial-grade (99.5%) material. Thus, the need to find a home for the extra glycerol may ultimately stymie the production of biodiesel fuel. See, generally, H. Noureddini et al., Adv. Environ. Res. 2 (1998) 232.

Ideally, the glycerol generated in biodiesel production could be returned to the fuel in the form of a compatible ether, thus adding another renewable component. Ethers have traditionally been added to fuels to improve combustion and reduce air pollution, and ethers based on glycerol have been suggested as potential fuel additives. U.S. Pat. No. 5,308,365, for example, teaches diesel or biodiesel fuel compositions that contain a mixture of di-tert-butyl and tri-tert-butyl ethers of glycerol. The reference teaches that, when used in diesel fuel, the glycerol ethers have good solubility, a high flash point, low water affinity, and negligible cetane reduction.

The di-tert-butyl ethers of glycerol are particularly valuable because they have low water solubility compared with monoalkyl ethers. Preferably, the amount of tri-tert-butyl ether produced is minimized because it consumes isobutylene without an added benefit versus the di-tert-butyl ethers. The preparation of di-tert-butyl ethers from glycerol and isobutylene using a soluble sulfonic acid catalyst (e.g., p-toluenesulfonic acid) has been previously described (see U.S. Pat. No. 5,476,971).

Solid catalysts, especially acidic ion-exchange resins, have also been used in the preparation of tert-butyl ethers from glycerol and isobutylene. For example, Karinen et al. (Appl. Catal. A 306 (2006) 128) teach the preparation of glycerol di-tert-butyl ethers using Amberlyst 35 resin. The authors conclude that a 3:1 molar ratio of isobutylene to glycerol favors production of di-tert-butyl ethers. Moreover, with Amberlyst 35, adding tert-butyl alcohol to the reaction mixture minimizes isobutylene dimerization and further improves selectivity to the diethers. β-Zeolites were not tested in or suggested for the process.

Dimerization (or oligomerization) of isobutylene in a process for making glycerol tert-butyl ethers is preferably minimized or eliminated. First, dimerization consumes isobutylene intended for ether production. Additionally, any diisobutylene reduces the flash point of a diesel or biodiesel fuel product.

Klepé\check{c}ová et al. (Appl. Catal. A 294 (2005) 141) teach to etherify glycerol using isobutylene or tert-butyl alcohol in the presence of acidic ion-exchange resins. The authors conclude that acidic macroreticular resins in dry form should be used because of their large pore diameter. They also teach that tert-butyl alcohol is less suitable than isobutylene for alkylation because the water formed in its dehydration deactivates the ion-exchange catalyst. The authors also tested various zeolites and showed that β-zeolite provides favorable selectivity to the di-tert-butyl ethers, although substantial isobutylene dimerization occurs, particularly at higher temperatures (i.e., 90° C.). The authors used CP814E, a product of Zeolyst, as the β-zeolite, which has a silica to alumina ratio of 25; this corresponds to a Si/Al molar ratio of 12.5. The reference does not teach to use a β-zeolite having a high Si/Al ratio and does not teach to use both isobutylene and tert-butyl alcohol in the etherification.

Similarly, Klepé\check{c}ová et al. (Chem. Pap. 60 (2006) 224) teach to etherify glycerol using tert-butyl alcohol catalyzed by ion-exchange resins. This reference suggests that large-pore zeolites can be used in the process, but only Amberlyst ion-exchange resins were tested. The authors conclude that tert-butyl alcohol is less desirable than isobutylene as an alkylating agent for glycerol.

In yet another paper, Klepé\check{c}ová et al. (Petrol. Coal 45 (2003) 54) compared the performance of Amberlyst resins to zeolites, including the β-zeolite CP814E, in the reaction of glycerol and tert-butyl alcohol. They concluded that the β-zeolite provides a favorable proportion of di-tert-butyl ether products. Isobutylene was not used as a reactant.

Honkela et al. (Catal. Lett. 87 (2003) 113) teaches that adding tert-butyl alcohol into an ion-exchange resin-catalyzed isobutylene dimerization process lowers conversion but improves selectivity to diisobutylene. No glycerol was included, and β-zeolites were not tested.

European Pat. Appl. No. 0 649 829 teaches a process for making tert-butyl ethers from glycerol and isobutylene using a β-zeolite catalyst. The catalysts have a Si/Al ratio greater than 5, preferably from 8.5 to 40. Example 3 shows good conversion of glycerol to products using isobutylene and a β-zeolite having a Si/Al ratio of 12.5. The diisobutylene content is reasonably low (11%) but is accompanied by relatively low (<60%) di-tert-butyl ether selectivity. The reference does not suggest any benefit of using a β-zeolite having a Si/Al ratio greater than 150 and does not teach to add tert-butyl alcohol into the process.

In sum, while β-zeolites have been used as catalysts in a process for making di-tert-butyl ethers from glycerol and isobutylene, the value of selecting β-zeolites having a high Si/Al molar ratio was overlooked. Moreover, while tert-butyl alcohol has been used as a less-favored alternative to isobutylene in such reactions, it has not been used in combination with isobutylene.

### SUMMARY OF THE INVENTION

In one aspect, the invention is a process for making glycerol di-tert-butyl ethers. The process comprises reacting glycerol and isobutylene in the presence of a β-zeolite having a silicon to aluminum ratio greater than 150. In another aspect, the invention is a process comprising reacting glycerol and isobutylene in the presence of a β-zeolite and added tert-butyl alcohol. Each process selectively provides glycerol di-tert-butyl ethers while reducing the generation of isobutylene oligomers. We surprisingly found that utilizing both aspects of the inventive process affords a di-tert-butyl ether product mixture containing less than 5 wt. % of isobutylene oligomers.

### DETAILED DESCRIPTION OF THE INVENTION

In the inventive process, glycerol and isobutylene react in the presence of a β-zeolite to produce a glycerol di-tert-butyl ether. Glycerol suitable for use in the invention comes from a variety of sources and need not have high purity. Most glycerol is obtained from natural sources, particularly animal and vegetable fats and oils as a by-product from the production of soap, fatty acids, or fatty esters (including the methyl esters used for biodiesel). Suitable glycerol includes synthetic glycerol produced from propylene or other starting materials. Also suitable for use is glycerin, a purified commercial product, which normally contains at least 95% glycerol, although different grades are commercially available. Prior to use in the inventive process, the glycerol can be refined, if desired, by distillation, carbon treatment, ion-exchange, steam-deodorization, bleaching, or other common techniques, and combinations thereof. In a preferred process of the invention, the glycerol is a by-product from a fat or oil, preferably one that is being converted into methyl esters for use as biodiesel fuel.

Isobutylene reacts with the glycerol. Suitable isobutylene is usually obtained from petroleum refinery and petrochemical complexes that crack petroleum fractions and natural gas liquids, particularly from catalytic, thermal, or steam cracking processes. It can also be produced by tert-butyl alcohol dehydration, olefin metathesis, ether cracking, isobutane dehydrogenation, or butene isomerization. Isobutylene is commercially available from many suppliers, and its purity level is typically not critical.

The isobutylene is normally used in excess compared with the amount of glycerol, and the amounts are generally adjusted to maximize the amount of glycerol di-tert-butyl ethers produced. Preferably, the molar ratio of isobutylene to glycerol ranges from 1.5:1 to 10:1, more preferably from 2:1 to 5:1, and most preferably from 2.5:1 to 3.5:1.

The reaction is performed in the presence of a β-zeolite. β-Zeolites are synthetic aluminosilicates having a well-defined three-dimensional structure of interconnecting channels. The most well-known member of the family is zeolite beta, which is also known as “BEA*.” Supplemental information is also available online from the International Zeolite Association. β-Zeolites having a variety of different Si/Al molar ratios are available commercially, and suitable β-zeolites are available from Zeolyst International and Süd-Chemie. Preferred β-zeolites are in the hydrogen, sodium, or ammonium form, more preferably the hydrogen form, and have surface areas within the range of 400 to 750 m²/g, more preferably 600 to 750 m²/g. The β-zeolite can be calcined prior to use. Calcination can be used, e.g., to convert the ammonium form of the β-zeolite to the hydrogen form. Calcination is preferably performed by heating the β-zeolite, typically for several hours, at temperatures greater than 100° C., preferably from 200° C. to 700° C., most preferably from 400° C. to 600° C. Suitable β-zeolites include these Zeolyst products: CP814E, CP814C, and CP811C-300; and Zeolite Beta from Süd-Chemie.

In one preferred process of the invention, the β-zeolite has a Si/Al molar ratio greater than 150, preferably greater than 200, more preferably greater than 250, and most preferably within the range of 200 to 500. A β-zeolite having a Si/Al molar ratio of about 300 is particularly preferred.

We surprisingly found that using a β-zeolite having a Si/Al molar ratio greater than 150 enables the selective production of glycerol di-tert-butyl ethers while minimizing the amount of isobutylene oligomerization. Preferably, the product mixture contains less than 15 wt. % of isobutylene oligomers. In particular, compare the results from Example 1 (13% diisobutylene (DIB) formed using a β-zeolite having a Si/Al=300) with Comparative Examples 4 and 7 (20-21% DIB formed using β-zeolites having Si/Al ratios of 150 or 25). The ability to influence the DIB level by adjusting the Si/Al ratio of the zeolite is unexpected and valuable.

The amount of β-zeolite used is not critical and depends on many factors, including the kind of process used (e.g., batch or continuous; stirred-tank or fixed-bed, etc.), the particular zeolite selected, reaction temperature and pressure, reaction time, and other considerations. It is convenient to use an amount within the range of 0.1 to 10 wt. %, preferably from 1 to 5 wt. %, and more preferably from 2 to 4 wt. % based on the combined amount of isobutylene and glycerol.

In another aspect of the invention, the reaction is performed in the presence of added tert-butyl alcohol. We surprisingly found that adding tert-butyl alcohol into the reaction of glycerol and isobutylene catalyzed by a β-zeolite dramatically reduces the amount of isobutylene oligomers formed, particularly diisobutylene and triisobutylene. The effect appears to be general for β-zeolites and is independent of the zeolite's Si/Al molar ratio. See Table 1, below, where the same β-zeolite is used with and without tert-butyl alcohol present. The reduction in the level of isobutylene oligomers is typically more than 50%.

The amount of tert-butyl alcohol used is not believed to be critical. Preferably, the amount ranges from 0.01 to 100 moles per mole of glycerol used. More preferably, the amount used ranges from 0.1 to 1 mole, and most preferably from 0.3 to 0.7 moles of tert-butyl alcohol per mole of glycerol. It is convenient and effective to use about 0.5 moles of tert-butyl alcohol per mole of glycerol.

In a preferred aspect, tert-butyl alcohol is used in combination with a β-zeolite having a Si/Al mole ratio greater than 150 (see Example 2, below). This enables the selective production of glycerol di-tert-butyl ethers while limiting the isobutylene oligomer content below 5 wt. %. Thus, while tert-butyl alcohol alone provides significant advantages for reducing the diisobutylene content, a further benefit results from selection of a β-zeolite having a high Si/Al mole ratio.

The reaction of glycerol and isobutylene can be performed at any convenient combination of temperature and pressure. Preferably, the temperature is within the range of 20° C. to 200° C., more preferably from 40° C. to 150° C., and most preferably from 60° C. to 100° C. Pressures vary depending upon isobutylene consumption. The pressure in a typical batch process normally peaks early, then declines as isobutylene is consumed in the process. Thus, the pressure normally varies in the range between 20 psig and 500 psig, more preferably from 40 psig to 200 psig.

Isobutylene can be supplied to the reactor by any desired method. It can be added in a single portion, incrementally, or continuously. In a convenient batch approach, all of the isobutylene is simply charged as a liquid to the sealed reactor in one portion. A continuous feed is more suitable for a CSTR or other continuous process.

The progress of the reaction can be monitored by any convenient method, such as, for example, gas chromatography, infrared spectroscopy, nuclear magnetic resonance, or other techniques. Gas chromatography provides a fast and convenient way to measure conversion of glycerol to the tert-butyl ethers.

When the reaction is reasonably complete, the β-zeolite is separated from the liquid reaction products by any convenient means, which may include gravity or vacuum filtration, decanting, centrifugation, or the like. The desired di-tert-butyl ether is frequently distilled to isolate it from any unreacted glycerol and from the other tert-butyl ethers.

The following examples merely illustrate the invention. Those skilled in the art will recognize many variations that are within the spirit of the invention and scope of the claims.

### Gas Chromatography Method

Samples are analyzed using a Hewlett-Packard 6890N gas chromatograph equipped with an autosampler and front-and-back flame-ionization detectors. Column: 60-m capillary; 1-um film thickness for the front and back inlets. Flow: 2.86 mL/min. Temperature program: 60° C. initially; hold 1 min; ramp at 10° C./min. to 260° C.; hold 4 min. Split flows, front inlet: 286 mL/min; back inlet: 114 mL/min.

### EXAMPLE 1

A 100-mL stainless-steel Parr reactor equipped with a thermocouple, mechanical stirrer, addition ports, and pressure-relief disc is charged with glycerol (10 g, 0.11 mol) and CP811C-300, a β-zeolite catalyst having a Si/Al molar ratio=300 (1.1 g, 3.4 wt. %, product of Zeolyst, calcined 4 h at 500° C. prior to use). After purging with nitrogen, the reactor is evacuated, liquid isobutylene (18.2 g, 0.324 mol) is added in one portion from a tared Pope vessel, and the reactor is sealed. The initial reactor pressure is less than about 10 psig. The reactor is heated to 85° C. using an external glycol bath, and the pressure rises to about 140 psig, then gradually declines as the isobutylene reacts to about 70 psig. After heating at 85° C. with stirring for 3 hours, the reactor contents are allowed to cool overnight.

The reactor is opened, and the mixture is filtered to remove the catalyst. Analysis of the liquid by gas chromatography shows the following proportion of glycerol-based products (wt. %): glycerol: 0.67; glycerol mono-tert-butyl ethers: 19.1; glycerol di-tert-butyl ethers: 78.6; glycerol tri-tert-butyl ether: 1.64.

The amounts of diisobutylene and triisobutylene are also determined. These weight percentages are based on the combined amount of unreacted glycerol, glycerol ethers, diisobutylene, and triisobutylene. For diisobutylene (DIB): 12.7; triisobutylene (TIB): 1.44. Table 1 summarizes the results.

### EXAMPLE 2

Example 1 is repeated, except that the reaction is performed in the presence of tert-butyl alcohol (4.0 g, 0.054 mol). GC analysis of the reaction product shows (wt. %): glycerol: 1.05; mono-tert-butyl ethers: 22.8; di-tert-butyl ethers: 75.6; glycerol tri-tert-butyl ether: 0.49; DIB: 4.70; TIB: 0.16. See Table 1.

### EXAMPLE 3

Example 2 is repeated, except that Zeolite Beta, a β-zeolite catalyst having a Si/Al molar ratio=150 (1.1 g, 3.4 wt. %, product of Siud-Chemie, calcined 4 h at 500° C. prior to use) is used. GC analysis of the product mixture provides the results that appear in Table 1.

### COMPARATIVE EXAMPLE 4

Example 3 is repeated except that no tert-butyl alcohol is added. See Table 1.

### EXAMPLE 5

Example 2 is repeated, except that CP814C, a β-zeolite catalyst having a Si/Al molar ratio=38 (1.1 g, 3.4 wt. %, product of Zeolyst, calcined 4 h at 500° C. prior to use) is used. Results appear in Table 1.

### EXAMPLE 6

Example 2 is repeated, except that CP814E, a β-zeolite catalyst having a Si/Al molar ratio=25 (1.1 g, 3.4 wt. %, product of Zeolyst, calcined 4 h at 500° C. prior to use) is used and the reaction proceeds for 2 hours instead of 3. See Table 1.

### EXAMPLE 7

Example 6 is repeated except that no tert-butyl alcohol is added and 3.6 wt. % of the catalyst is used. See Table 1.

Table 1 shows that β-zeolite catalysts generally provide a high proportion of glycerol di-tert-butyl ethers. Higher ratios generally correspond to higher Si/Al ratios. Importantly, the amount of isobutylene oligomers is reduced significantly by selecting a β-zeolite having a Si/Al molar ratio greater than 150. Compare the results from Example 1 (about 13% diisobutylene using Si/Al=300) with those of Comparative Examples 4 and 7 (20-21% DIB using Si/Al=150 or 25).

Additionally, the results demonstrate the advantage of including tert-butyl alcohol (TBA) in the process. With TBA and a β-zeolite with a high Si/Al ratio (see Example 2), the isobutylene oligomer level can be suppressed to less than 5%. TBA helps to reduce the amount of isobutylene oligomers produced for β-zeolites generally. Compare their levels in Example 2 vs. Example 1; Example 3 vs. Comparative Example 4; and Example 6 vs. Comparative Example 7.

### EXAMPLE 8

A two-gallon, stainless-steel autoclave reactor equipped with a magnetic drive mixer, charge ports, thermocouple, Sterlco circulated hot oil heater, Athena high-temperature interlock, and pressure-relief disk is charged with glycerol (1000 g), tert-butyl alcohol (402 g), and CP811C-300 β-zeolite catalyst (110 g, product of Zeolyst, Si/Al molar ratio=300, calcined 4 h at 500° C. prior to use). The reactor is evacuated and isobutylene (1824 g) is introduced into the reactor. The contents are heated at 85° C. with stirring for 3 hours, then cooled to room temperature. GC analysis of the liquid phase shows (wt. %): glycerol: 2.0; mono-tert-butyl glycerol: 26; di-tert-butyl glycerol: 71; tri-tert-butyl glycerol: 0.26; diisobutylene: 3.8.

### EXAMPLE 9

Example 8 is repeated except that tert-butyl alcohol is omitted. GC analysis of the product shows (wt. %): glycerol: 0.29; mono-tert-butyl glycerol: 15; di-tert-butyl glycerol: 82; tri-tert-butyl glycerol: 2.4; diisobutylene: 17.

TABLE 1
Preparation of Di-Tert-Butyl Glycerols from Glycerol and Isobutylene
Using β-Zeolite Catalysts

| | | | | | Glycerol | | | Isobutylene | |
|---|---|---|---|---|---|---|---|---|---|
| | TBA | β-Zeolite | Si/Al | | tert-Butyl Ethers* | | | oligomers** | |
| Ex. # | used? | Catalyst | (molar) | Glycerol* | Mono- | Di- | Tri- | DIB | TIB |
| 1 | no | CP811C-300¹ | 300 | 0.67 | 19.1 | 78.6 | 1.64 | 12.7 | 1.44 |
| 2 | yes | CP811C-300 | 300 | 1.05 | 22.8 | 75.6 | 0.49 | 4.70 | 0.16 |
| 3 | yes | Zeolite Beta² | 150 | 1.08 | 19.1 | 78.0 | 1.79 | 9.44 | 0.15 |
| C4 | no | Zeolite Beta | 150 | 1.13 | 22.8 | 72.4 | 3.72 | 21.0 | 3.89 |
| 5 | yes | CP814C¹ | 38 | 1.17 | 24.3 | 74.1 | 0.40 | 12.0 | 2.14 |
| 6 | yes | CP814E¹ | 25 | 1.96 | 28.7 | 69.1 | 0.25 | 7.94 | 1.33 |
| C7 | no | CP814E | 25 | 2.70 | 26.0 | 70.6 | 0.72 | 20.0 | 3.75 |

¹Product of Zeolyst International.
²Product of Süd-Chemie.
TBA = tert-butyl alcohol;
DIB = diisobutylene;
TIB = triisobutylene.
*Wt. % based on glycerol + glycerol tert-butyl ethers only; determined by gas chromatography (GC).
**Wt. % based on glycerol + glycerol tert-butyl ethers + isobutylene oligomers; determined by GC.

Examples 8 and 9 demonstrate that the advantages seen using a β-zeolite having a high Si/Al ratio, particularly in the presence of added tert-butyl alcohol, can be achieved in a larger-scale process.

The preceding examples are meant only as illustrations. The following claims define the invention.

We claim:

1. A process which comprises reacting glycerol and isobutylene in the presence of a β-zeolite having a silicon to aluminum ratio greater than 150 to give a product mixture comprising a glycerol di-tert-butyl ether.

2. The process of claim 1 wherein the β-zeolite has a silicon to aluminum ratio within the range of 200 to 500.

3. The process of claim 1 wherein the reaction is performed in the presence of added tert-butyl alcohol.

4. The process of claim 1 wherein the reaction is performed at a temperature within the range of 30° C. to 150° C.

5. The process of claim 1 wherein the product mixture comprises glycerol tert-butyl ethers, at least 75 wt. % of which is the di-tert-butyl ether.

6. The process of claim 1 wherein the product mixture comprises less than 15 wt. % of isobutylene oligomers.

7. A process which comprises reacting glycerol and isobutylene in the presence of a β-zeolite and added tert-butyl alcohol to give a product mixture comprising a glycerol di-tert-butyl ether.

8. The process of claim 7 wherein the β-zeolite has a silicon to aluminum ratio within the range of 200 to 500.

9. The process of claim 8 wherein the product mixture comprises less than 5 wt. % of isobutylene oligomers.
